(19) United States
(12) Patent Application Publication
(10) Pub. No.: US 2013/0173006 A1
(43) Pub. Date: Jul. 4, 2013

(54) ADAPTABLE SHOULDER PROSTHESIS CUP

(71) Applicant: Marc Duport, Toulouse (FR)

(72) Inventor: Marc Duport, Toulouse (FR)

(21) Appl. No.: 13/823,323

(22) PCT Filed: Sep. 15, 2011
(86) PCT No.: PCT/FR2011/052116

§ 371 Date: Mar. 14, 2013

(30) Foreign Application Priority Data
Sep. 17, 2010 (FR) 10/57436

(51) Int. Cl. A61F 2/40

(52) U.S. Cl. CPC A61F 2/4081
USPC 623/19.11

(57) ABSTRACT

The present invention relates to a prosthesis cup (1), particularly a shoulder prosthesis cup, comprising a shell (2) shaped substantially as a hollow spherical cap delimiting a concave inner surface (3), the shell (2) having a coupling stem (4) that extends from this inner surface (3) and is designed to permit coupling of the cup (1) to a fixing element, particularly an anchoring screw (26) or an anchoring pin (13), the cup (1) being characterized in that it additionally comprises anchoring and/or coupling means with a wall (6) which extends from the inner surface (3) of the shell (2) and has a substantially tubular shape and which, together with the coupling stem (4), delimits a recess (8) having what is substantially a ring shape.

### TECHNICAL FIELD

The invention relates to a shoulder cup, in particular a shoulder prosthesis cup.

### RELATED ART

In France, approximately 15,000 shoulder prostheses are placed each year, including total shoulder prostheses, called standard prostheses, reverse prostheses, and more recently resurfacing prostheses.

This figure, which shows a clear increase in recent years, is in particular due to the increase in the number of pathologies for which the placement of a shoulder prosthesis is now indicated.

In this context, prosthetic shoulder surgery remains a very specialized, highly technical surgery, requiring a long learning period and the use of ancillaries that are often complex. It is therefore desirable to simplify the prostheses and ancillaries so as to facilitate the procedures, make it more reproducible, and therefore decrease postoperative complications. Such a simplification of the ancillary and the procedure may also have the advantage of decreasing the costs of those operations to place shoulder prostheses.

Several types of shoulder prosthesis exist. Certain traditional prostheses, like that described in US 2004/153161, are fixed on the humerus, using a humeral rod, after a resulting resection of the bone. The installation of a humeral rod requires a significant recess in the bone to receive it, thereby decreasing the bone capital and making the bone more fragile. The placement of such a prosthesis is long and complex. After the rod is installed in the recess, the space between the bone and the humeral rod is generally filled with cement. With time, the cement may become dislocated and no longer ensure correct fixing of the rod.

Resurfacing prostheses also exist, like that described in FR 2,928,827, which are placed by epiphysis of the humerus after potential light planing of the surface of the epiphysis. Such prostheses are not necessarily anchored in the bone as previously described.

When a simple resurfacing of the epiphysis is not indicated, for example if the bone is too fragile, the surgeon may choose to use an intermediate so-called “half-resurfacing” technique where part of the epiphysis is resected. The surgeon is then called upon to use a prosthesis like that described in document EP 0,538,895, including a cup designed to replace the part of the epiphysis that is resected and an anchoring screw ensuring fastening of the cup on the bone.

Document US 2003/0163202 describes a cup including a central fastening stem.

It is also known from document WO 2008/146124 to replace the anchoring screw previously described with a substantially cylindrical anchoring pin. The cup is then provided with a coupling stem cooperating by interlocking with the cavity, with a complementary cylindrical shape, formed to that end in the anchoring pin. Such a cup is then kept in place owing to the pressure exerted by the glenoid. However, the nature of the coupling of the cup and the anchoring pin does not make it possible to avoid rotation of the cup after it is implanted, which can cause problems. Furthermore, the cup simply being interlocked in the cavity of the anchoring pin, the anchoring of the cup on the humeral head may prove insufficient, and thereby cause pain or discomfort for the patient.

Document FR 2,686,503 describes a rehabilitation prosthesis for a femoral head, including a cup equipped with a central anchoring stem in the bone, surrounded by several spurs that are parallel thereto and are designed to penetrate the bone of the femur to improve the fastening of the cup.

All of the known cups are specifically suitable for a specific surgical technique. The surgeon must therefore learn the different techniques and must have several ancillaries adapted to each technique. Furthermore, if, the surgeon encounters difficulties during an operation, it is not possible for him to adapt the prosthesis, for example by changing the implantation method of the cup.

### BRIEF SUMMARY

The present invention aims to resolve all or some of the various aforementioned drawbacks.

In this context, the present invention aims to propose an adaptable cup making it possible to use different techniques (traditional, resurfacing, and semi-resurfacing), which preserves the bone capital of the patient and which is easy and quick to place.

To that end, the present invention relates to a prosthesis cup, particularly a shoulder prosthesis cup, comprising a shell substantially shaped as a hollow spherical cap delimiting a concave inner surface, the shell having a coupling stem that extends from this inner surface and is designed to permit coupling of the cup to a fixing element, particularly an anchoring screw or an anchoring pin, the cup being characterized in that it additionally comprises anchoring and/or coupling means with a wall which extends from the inner surface of the shell and has a substantially tubular shape and which, together with the coupling stem, delimits a recess having what is substantially a ring shape.

Thus, a cup according to the invention is adaptable: The coupling stem makes it possible to couple the cup to a fastening element depending on the surgeon's needs. The anchoring and/or coupling means make it possible on the one hand to anchor the cup directly in the bone to improve its fastening, and on the other hand, depending on the needs, to couple the cup with an intermediate member having a shape in the form of a ring portion and suitable for being housed in the recess delimited between the anchoring and/or coupling means and the coupling stem. Such an intermediate member may for example be a coupling member between the cup and an anchoring screw or an intermediate bush making it possible to space the cup away from the bone for better adaptability of the prosthesis on the patient. The anchoring and/or coupling means therefore allow the surgeon to choose the technique he will use to fasten the cup on the patient and potentially change it during operation. In light of the possible compatibility between the anchoring pin and the intermediate member, the surgeon also has the possibility of using both at the same time.

The ring-shaped recess, delimited by the tubular wall, allows simple and fast assembly and fastening of an intermediate member, such as a bush making it possible to increase the height of the ring. To facilitate fastening of an intermediate member, the ring may form a Morse taper.

According to one feature of the invention, the wall is serrated. A serrated wall makes it possible to improve the anchoring of the cup in the bone when the anchoring and/or coupling means are used to anchor the cup. The separation also makes it possible to avoid rotation of the cup after it is inserted in the bone. Lastly, the serrated wall prevents the rotation of any intermediate member that may be used.

According to one embodiment, the wall has a thickness that decreases as it moves away from the inner surface. The decrease in the thickness of the wall moving away from the inner surface gives the wall an improved penetration capacity in the bone.

According to one embodiment, the substantially tubular wall has at least one longitudinal slot. Such a longitudinal slot makes it possible to improve the anchoring of the cup in the bone.

According to one embodiment, the recess is configured to receive an intermediate member having a substantially tapered outer surface.

According to one embodiment, the coupling stem and the anchoring and/or coupling means are concentric, the coupling stem extending from the pole of the shell.

According to one possibility, the shell also includes at least one anchoring rib formed on the inner surface. Each anchoring rib increases the anchoring of the cup in the bone. In particular, when the cup is coupled to a coupling member and the anchoring and/or coupling means are not used to anchor the cup, each anchoring rib ensures fastening thereof. Each anchoring rib also makes it possible to avoid the rotation of the cup once it is placed on the patient and provides the cup with tractive holding power once it is implanted.

According to one embodiment, each anchoring rib is configured to cause the rotation of the cup when it is placed on a bone. Thus, the rotation of the cup during its placement makes it possible to improve the penetration of the anchoring and/or coupling means in the bone, when they are used to fasten the cup. Once the cup is implanted, each anchoring rib prevents the cup from rotating and opposes the pulling out of the cup.

According to one possibility, the shell also has at least one notch arranged on the edge of the shell. Such a notch is useful to fasten an ancillary on the cup designed to maintain the cup during impaction. Preferably, said ancillary is designed to impart a rotation of 10° to the cup by cooperating with said notches.

The present invention lastly relates to a kit comprising at least one cup according to the invention and at least one fastening element, in particular an anchoring screw or an anchoring pin and/or at least one intermediate member having a ring-shaped portion and suitable for being housed in the recess delimited between the anchoring and/or coupling means and the coupling stem.

### BRIEF DESCRIPTION OF THE DRAWINGS

The invention will be well understood using the detailed description thereof below in light of the appended figure, showing one embodiment of the cup as a non-limiting example, in which:

FIG. 1 is a diagrammatic perspective view of a cup;

FIG. 2 is a diagrammatic perspective view of the cup of FIG. 1 coupled with an anchoring pin;

FIG. 3 is a diagrammatic perspective view of the cup of FIG. 1 coupled with an intermediate bush and an anchoring pin;

FIG. 4 is a diagrammatic perspective view of the cup of FIG. 1, a coupling member and an anchoring screw for fastening the prosthesis.

### DETAILED DESCRIPTION

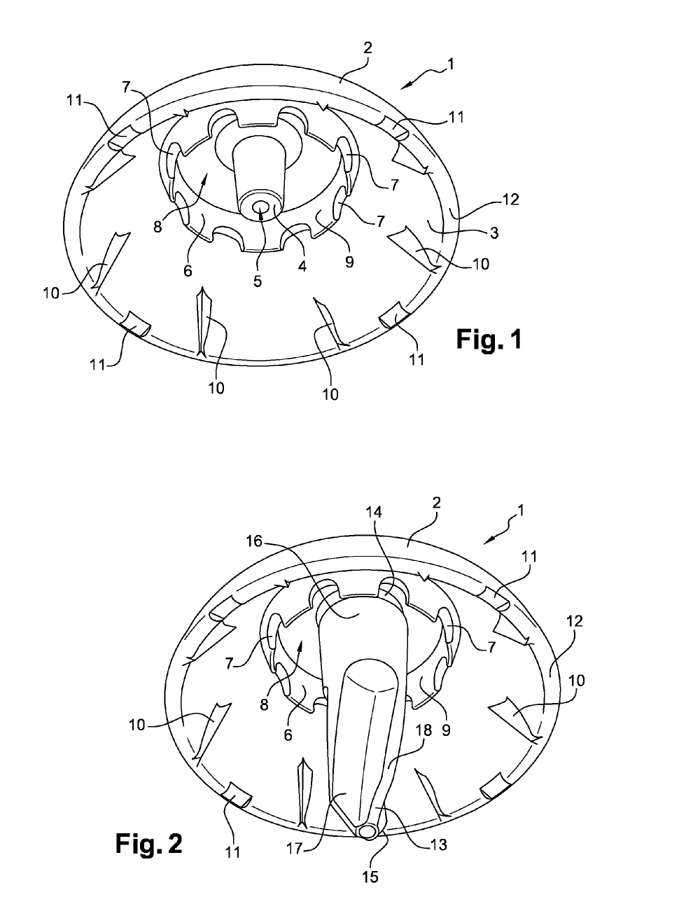

A prosthesis cup 1, illustrated in FIG. 1, includes a shell 2 substantially in the form of a hollow spherical cap. The shell 2 delimits a concave inner surface 3 and has a coupling stem 4 that extends from that inner surface 3 at the pole of the shell 2. The coupling stem 4 is, for example, provided with a through opening 5 extending along the axis of the spherical cap. The cup 1 also includes anchoring and/or coupling means such as a wall 6 that extends from the inner surface 3 and has a substantially tubular shape. This wall 6 includes eight gaps 7 formed at the free end thereof. Of course, the number of gaps 7 can be adapted as needed or depending on the size of the cup 2. Likewise, the shape of each gap 7 is adaptable and can, for example, be rounded, as illustrated in FIG. 1, or rectangular. Of course, the depth of each gap 7 may vary until it is substantially equal to the height of the wall 6. The thickness of the wall 6 decreases as it moves away from the inner surface 3. The wall 6 is arranged concentrically with the coupling stem 4, such that they delimit a recess 8 substantially in the shape of a ring. The face 9 of the wall 6 arranged across from the coupling stem 4 has a female tapered shape.

The cup 1, illustrated in FIG. 1, also has eight anchoring ribs 10 formed on the inner surface 3. Of course, the number of anchoring ribs 10 may be adapted as needed or depending on the size of the cup 2. Each anchoring rib 10 extends along a unique longitudinal axis, off-centered relative to the axis of the spherical cap.

Lastly, the cup 1, illustrated in FIG. 1, for example has four notches 11 arranged on the edge 12 of the shell 2.

The surgeon can implant the cup 1 in several ways outlined below, as needed.

The surgeon first has the option of using an anchoring pin 13, illustrated in FIG. 2, to implant the cup 1, for example in the context of resurfacing of the epiphysis of the bone.

The anchoring pin 13 extends along the longitudinal axis between a first end 14, designed to cooperate with the cup 1, and a second end 15, designed to cooperate with the bone. It has a generally tapered shape. The anchoring pin 13 includes a first portion 16 designed to allow coupling of the cup 1. This first portion 16 has a coupling orifice, not shown, extending along the longitudinal axis of the pin 13 and emerging on the first end 14. The shape of this coupling orifice is complementary to the shape of the coupling stem 4 of the cup 1, so as to allow interlocking thereof in the coupling orifice of the pin 13 to couple the cup 1 and pin 13. Advantageously, the coupling stem 4 and the coupling orifice are tapered. For example, the coupling stem 4 forms a male Morse cone and the coupling orifice is a complementary female Morse cone.

The pin 13 also includes a second portion 17 designed to anchor the pin 13 in a bone. The second portion 17 to that end has three sharp helical edges 18 allowing the pin 13 to penetrate the bone like a drill. The sharp helical edges 18 impart a rotational movement to the pin 13 when they penetrate the bone, ensuring better anchoring. The sharp helical edges 18 and the ribs 10 are configured to impart a rotation to the cup 1 in a same direction: during the impaction, synergy appears between the sharp helical edges 18 and the ribs 10 that cause a rotational movement of approximately 10°.

When the anchoring pin 13 is used to fasten the cup 1, the surgeon couples the cup 1 on the pin 13, inserting the coupling stem 4 into the coupling orifice of the pin 13, then fastens the pin 13 in the epiphysis of the bone. The impaction of the pin 13 and the cup 2 is continued until the wall 6 of the anchoring and/or coupling means is pushed into the epiphysis of the bone to ensure additional anchoring of the cup 1.

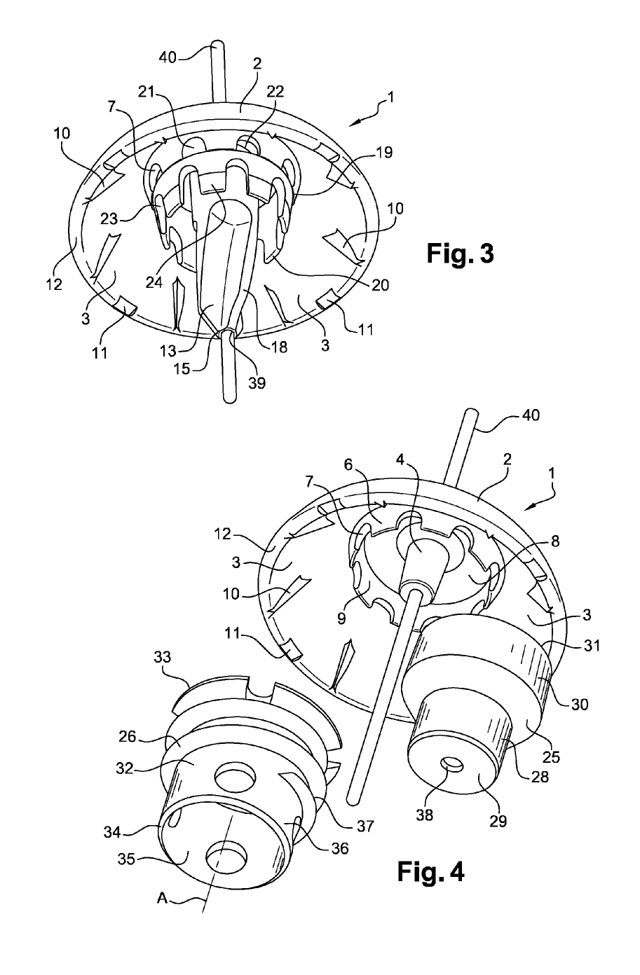

The wall 6 has a height not exceeding 10% of the radius of the spherical cap. Preferably, the wall 6 has a height smaller than 7 mm. When the epiphysis of the bone has been partially or completely resected, the height of the wall 6 may be insufficient for the anchoring and/or coupling means to be anchored in the bone. The surgeon then has the possibility of using an intermediate bush 19 illustrated in FIG. 3. Such an intermediate bush 19 has a tubular body 20 extending along a longitudinal axis between two opposite ends. The tubular body 20 comprises a coupling portion 21 with a shape suitable for being inserted into the recess 8 of the cup 1, such that the outer surface 22 of the coupling portion 21 is in contact with the face 9 of the wall 6 arranged across from the coupling stem 4 to ensure coupling without play of the cup 1 and the intermediate bush 19. The coupling portion 21 may optionally include a stop, not shown, protruding from the outer surface 22, said stop being designed to cooperate with one of the gaps 7 of the cup 1 to block the rotation of the intermediate bush 19 in the recess 8.

The intermediate bush 19 also has an anchoring portion 23 including an extension wall 24 having a shape similar to the wall 6. Thus, the extension wall 24 constitutes an extension of the wall 6 and is designed to be fixed in the bone in the same way as the wall 6. When the intermediate bush 19 is coupled with the cup 1, the free end of the intermediate bush 19 is situated at a distance from the pole of the spherical cap comprised between 90% and 100% of the radius thereof, such that the free end of the intermediate bush 19 is flush with the edge 12 of the shell 2. Thus, the total ablation of the cup 1 is possible and simple, since one need only saw the bone using a saw guided by the edge 12 of the shell 2: the intermediate bush 19 is then not sectioned by the saw and may be completely removed with the cup 1.

The surgeon can also use a coupling member 25 and an anchoring screw 26 to fasten the cup, illustrated in FIG. 4.

The coupling member 25 includes a first portion 27 with a substantially tapered shape having a male intermediate mounting portion 28 forming a male Morse cone 29. The coupling member 25 also includes a second tubular portion 30 provided with a cavity, not shown, that is substantially tapered and delimiting a female intermediate mounting portion. The female intermediate mounting portion forms a female Morse cone, not shown, sized to cooperate by shape matching with the coupling stem 4 of the cup 1. According to the embodiment shown in the figures, the coupling member 25 has a male Morse cone 29 and a female Morse cone whereof the respective axes of symmetry are combined. Alternatively and not shown, the male Morse cone 29 and the female Morse cone may have respective axes of symmetry that are distinct and offset and/or not parallel. The outer surface 31 of the second tubular portion 30 has a shape adapted to be inserted into the recess 8 of the cup 1, such that the outer surface 31 of that second tubular portion 30 is in contact with the face 9 of the wall 6 arranged across from the coupling stem 4 to ensure coupling without play of the cup 1 and the coupling member 25.

The anchoring screw 26, illustrated in FIG. 4, includes a tubular body 32 extending along the longitudinal axis A between a proximal end 33, designed to cooperate directly or indirectly with the cup 1 or the coupling member 25, and an opposite distal end 34, designed to be fixed in the epiphysis of the bone. The tubular body 32 delimits an inner peripheral surface 35, illustrated in FIG. 4, and an outer peripheral surface 36 provided with screwing means, for example made in the form of a thread 37 to allow screwing of the screw 26 in the bone.

The inner peripheral surface 35 delimits a female mounting portion, not shown, that extends substantially from the proximal end 33 and is sized to cooperate by shape matching with the male Morse cone 29 of the coupling member 25. In one embodiment not shown in the figures, the female mounting portion is sized to cooperate by shape matching with the coupling stem 4 of the cup 1 and to be directly inserted into the recess 8.

Thus, the anchoring screw 26 described above makes it possible to fix the cup 1. When the tubular body 32 of the screw 26 is fixed in the bone, the correct anchoring of the screw 26 is ensured owing to the threading 37 and the placement procedure is simplified, since the coupling of the cup 1 on the screw 26 directly or indirectly with the coupling member 25 is done by simple interlocking.

In order to facilitate the positioning of the cup 1 and the anchoring pin 13 or the coupling member 25, the latter each include a through orifice 38, 39 extending along the longitudinal axis of the coupling member 25 or the anchoring pin 13, respectively. Thus, irrespective of the method used to implant the cup 1, the surgeon has the option of using a rod 40, illustrated in FIGS. 3 and 4, to align the cup 1 and the anchoring pin 13 or the coupling member 25 on the epiphysis. The rod 40 is first inserted into an orifice formed to that end in the bone to guide and guarantee the correct orientation of the cup 1. The anchoring pin 13 or the coupling member 25 and the cup are then simply slid onto the rod 40 using their respective through orifices 5, 39, 38.

Thus, the cup 1 according to the invention described above is greatly adaptable to the different surgical techniques commonly used to place a shoulder prosthesis. The anchoring and/or coupling means make it possible to fix the cup 1 directly in the bone. They also make it possible, depending on the surgeon's needs, to couple the cup 1 to a coupling member 25 or an anchoring screw 26 in the shape of a ring portion suitable for being housed in the recess 8 delimited between the anchoring and/or coupling means and the coupling stem 4.

Lastly, the cup 1, the anchoring pin 13, the coupling member 25, the intermediate bush 19 and/or the screw 26 are advantageously covered, in whole or in part, with a layer of material favoring bone integration and bone growth, for example such as a layer of hydroxyapatite.

Of course, the example embodiment provided above is in no way limiting, and other details and improvements may be made to the cup 1 according to the invention without going beyond the scope of the invention, where other cup forms may be produced.

1. A prosthesis cup, particularly a shoulder prosthesis cup, comprising a shell substantially shaped as a hollow spherical cap delimiting a concave inner surface, the shell having a coupling stem that extends from this inner surface and is designed to permit coupling of the cup to a fixing element, particularly an anchoring screw or an anchoring pin, the cup further comprising anchoring and/or coupling means with a wall-which extends from the inner surface of the shell and has a substantially tubular shape and which, together with the coupling stem, delimits a recess having what is substantially a ring shape.

2. The cup according to claim 1, wherein the wall is serrated.

3. The cup according to one of claim 1, wherein the wall has a thickness at decreases as it moves away from the inner surface.

4. The cup according to claim 1, wherein the recess is configured to receive an intermediate member having a substantially tapered outer surface.

5. The cup according to claim 1, wherein the coupling stem and the anchoring and/or coupling means are concentric, the coupling stem extending from the pole of the shell.

6. The cup according to claim 5, wherein the shell also includes at least one anchoring rib formed on the inner surface.

7. The cup according to claim 6, wherein each anchoring rib is configured to cause the cup to rotate when it is placed on a bone.

8. The cup according to claim 1, wherein the shell also has at least one notch arranged on the edge of the shell.
